(19) United States
(12) Patent Application Publication
(10) Pub. No.: US 2013/0231658 A1
(43) Pub. Date: Sep. 5, 2013

(54) EXPANDABLE ABLATION DEVICE AND METHODS FOR NERVE MODULATION

(71) Applicant: BOSTON SCIENTIFIC SCIMED, INC., Maple Grove, MN (US)

(72) Inventors: HUISUN WANG, MAPLE GROVE, MN (US); JAMES M. ANDERSON, FRIDLEY, MN (US)

(73) Assignee: BOSTON SCIENTIFIC SCIMED, INC., MAPLE GROVE, MN (US)

(21) Appl. No.: 13/783,016

(22) Filed: Mar. 1, 2013

Related U.S. Application Data
(60) Provisional application No. 61/605,583, filed on Mar. 1, 2012.

(51) Int. Cl. A61B 18/14

(52) U.S. Cl. CPC A61B 18/1492
USPC 606/41

(57) ABSTRACT

Medical devices for nerve modulation through the wall of a blood vessel are disclosed. The medical device may include an elongate member having a proximal end and a distal end. A hollow ablation member may be disposed at the distal end of the elongate member and includes a number of electrodes positioned on its outer surface. The ablation member may be configured to shift between a collapsed position and an expanded position such that that a portion of the ablation member can be brought into contact with the wall of the blood vessel or placed adjacent to the wall of the blood vessel. The ablation member may also be retractable from the blood vessel treatment site.

### CROSS-REFERENCE TO RELATED APPLICATIONS

This application claims priority under 35 U.S.C. §119 to U.S. Provisional Application Ser. No. 61/605,583, filed Mar. 1, 2012, the entirety of which is incorporated herein by reference.

### TECHNICAL FIELD

The present disclosure relates to methods and apparatuses for modulating nerves through the walls of blood vessels. Such modulation may include ablation of nerve tissue or other modulation technique.

### BACKGROUND

Certain treatments require temporary or permanent interruption or modification of select nerve functions. One example treatment is renal nerve ablation, which is sometimes used to treat conditions related to congestive heart failure. The kidneys produce a sympathetic response to congestive heart failure, which among other effects, increases the undesired retention of water and/or sodium. Ablating some nerves running to the kidneys may reduce or eliminate this sympathetic function, providing a corresponding reduction in the associated undesired symptoms.

Many nerves (and nervous tissue such as brain tissue), including renal nerves, run along the walls of or in close proximity to blood vessels and these nerves can be accessed intravascularly through the blood vessel walls. In some instances, it may be desirable to ablate perivascular renal nerves using a radio frequency (RF) electrode. Such treatment, however, may result in thermal injury to the vessel at the electrode and other undesirable side effects such as, but not limited to, blood damage, clotting, and/or protein fouling of the electrode. To prevent such undesirable side effects, some techniques attempt to increase the distance between the vessel walls and the electrode. In these systems, however, the electrode may inadvertently contact the vessel walls, causing irreparable damage.

Therefore, there remains room for improvement and/or alternatives in providing systems and methods for intravascular nerve modulation.

### SUMMARY

The disclosure is directed to several alternative designs and methods of using medical device structures and assemblies.

Accordingly, some embodiments pertain to a medical device for nerve modulation through the wall of a blood vessel. The medical device includes an elongate member having a proximal end and a distal end. Further, a hollow ablation member is disposed at the distal end of the elongate member. The ablation member includes a number of electrodes positioned on its outer surface. In addition, the ablation member is configured to switch between a collapsed state and an expanded state such that that a portion of the ablation member may be brought into contact with a wall of a blood vessel. The ablation member may be self-expandable or expanded by an actuating means. For example, the ablation member may be implemented as a stent. The ablation member may further include an insulated section. The insulated section may cover the outer surface of the ablation member or may partially surround the electrodes. Alternatively, the ablation member may be made of a non-conductive material. The medical device may further include one or more sensors and the electrodes may be coupled to one or more conductors.

Some other embodiments pertain to a system for nerve modulation through the wall of a blood vessel. The system includes a sheath having a proximal end, a distal end, and a lumen extending from the proximal to distal end. An elongate member extends along a central elongate axis within the lumen of the sheath, the elongate member having a proximal end and a distal end. The system further includes an expandable hollow ablation member coupled to the distal end of the elongate member. The ablation member may include an insulating section, and a number of electrodes disposed on its outer surface. The ablation member may be configured to switch between a collapsed state, and an expanded state in which the ablation member extends out of the distal end of the sheath such that that a portion of the ablation member contacts the walls of the blood vessel. The ablation member is self-expandable or expanded by an actuating means. In one aspect, the ablation member may be a stent, or other hollow tubular member. In addition, the insulated section may cover the outer surface of the ablation member or partially surround the electrodes. Alternatively, the ablation member may be made of a non-conductive material. Also, the system may include one or more sensors.

Some embodiments pertain to a method for ablating a renal nerve through a blood vessel. The method includes advancing a medical device proximate to a desired location in a vessel lumen. The medical device includes an elongate member having a proximal end and a distal end. Further, a hollow ablation member is disposed at the distal end of the elongate member. The ablation member includes a number of electrodes positioned on its outer surface. In addition, the ablation member is configured to switch between a collapsed state, and an expanded state such that that a portion of the ablation member contacts the walls of the blood vessel. The method further includes deploying the expandable ablation member by reconfiguring the ablation member to its expanded state in the vessel lumen, and activating one or more electrodes to ablate at least a portion of the nerve tissue.

The summary of some example embodiments is not intended to describe each disclosed embodiment or every implementation of the disclosure.

### BRIEF DESCRIPTION OF THE DRAWINGS

The present disclosure may be more completely understood in consideration of the following detailed description of various embodiments in connection with the accompanying drawings, in which:

FIG. 1 is a cross-sectional side view of one embodiment of a renal nerve modulation system.

FIG. 2 is a sectional side view of one embodiment of an electrode used in the renal nerve modulation system.

FIG. 3 is a three dimensional view of an embodiment of a renal nerve modulation system with a stent having insulation on the stent.

FIG. 4A is a three dimensional view of the renal nerve modulation system shown in FIG. 1, in an expanded state.

FIG. 4B is a three dimensional view of the renal nerve modulation system shown in FIG. 1, in a collapsed state.

FIG. 5 illustrates an embodiment of the renal nerve modulation system shown in FIG. 1, disposed within a blood vessel in its expanded state.

FIG. 6 illustrates the distal portion of an embodiment of a renal nerve modulation system in its expanded state.

While specific embodiments of the present disclosure have been shown in the drawings and are discussed in detail below, the implementation of the disclosure is amenable to various modifications and alternative forms. It should therefore be understood that the intention is not to limit aspects of the disclosure to the particular embodiments described. On the contrary, the intention is to cover all modifications, equivalents, and alternatives falling within the spirit and scope of the present disclosure.

### DETAILED DESCRIPTION

For the following defined terms, these definitions shall be applied, unless a different definition is given in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many of which will deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation for treatment of hypertension, it is contemplated that the devices and methods may be used in other applications where nerve modulation and/or ablation are desired.

The present disclosure provides methods and systems to ablate a renal nerve. To this end, the system may employ an expandable stent-like structure having electrodes on its outer surface. In general, the stent-like structure has a cylindrical shape and assumes a collapsed state during insertion and retrieval, and once deployed, the stent expands to contact the blood vessel walls. Electrodes may be positioned on the surface of the stent in a suitable manner, as desired. The self-expanding stent described in the present disclosure provides substantially uniform contact between the electrodes and the vessel wall. The term stent is used herein to indicate a tubular expandable structure that may be self-expanding or may be expanded by other means (e.g. a balloon) to a larger diameter; the term stent is not intended to reference the structures of the same name that may be implanted during angioplasty procedures and like procedures to expand an occluded blood vessel.

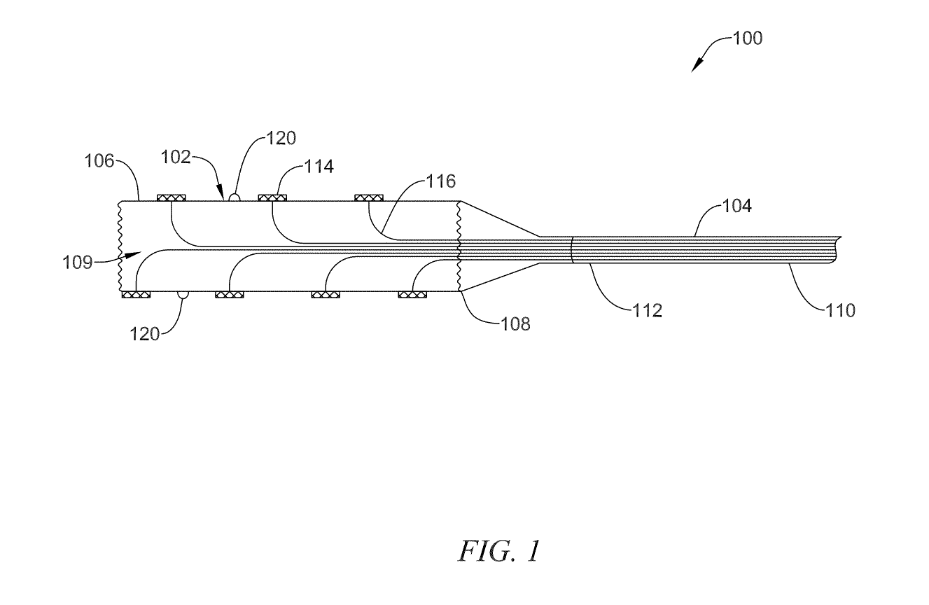

FIG. 1 is a cross-sectional view of an exemplary renal nerve modulation system 100 that includes an ablation member configured as a stent 102 and an elongate member 104. The stent 102 includes a distal end 106, a proximal end 108, and a lumen 109 extending between the distal and proximal ends 106, 108. Further, the elongate member 104 includes a proximal end 110, and a distal end 112, which is connected to the proximal end 108 of the stent 102. For ablation purposes, one or more electrodes 114 may be mounted on the exterior surface of the stent 102.

In general, the elongate member 104 may be a tubular member extending proximally from the proximal end of the stent 102, the proximal end 110 of the elongate member 104 being configured to remain outside a patient's body. The proximal end of the elongate member 104 may include mechanisms for controlling the electrodes 114 or for facilitating various treatments.

The elongate member 104 may be made of any suitable biocompatible material such as polyurethane, plastic, or any other such material. Moreover, the elongate member 104 may be flexible along its entire length or adapted for flexure along portions of its length. Alternatively, the elongate member's distal end may be more flexible while the remaining member may be stiffer. Flexibility allows the elongate member 104 to maneuver in the circuitous vasculature, while stiffness allows the required force to be transmitted to urge the elongate member 104 forward. The diameter of the elongate member 104 may vary according to the desired application, but it is generally smaller than the typical diameter of a patient's vasculature.

The stent 102 along with the elongate member 104 may be configured to be advanced into a body lumen such as a renal artery to ablate body tissue (e.g., renal nerves or ganglia). The stent 102 is implemented as a hollow, elongate tube with cross-sectional configuration adapted according to a desired body lumen. In the illustrated embodiment, the stent 102 is generally circular, with a generally circular hollow interior lumen 109. The interior lumen 109 may have an open distal end and/or an open proximal end. In some embodiments, the stent 102 has an open proximal end and an open distal end to allow for blood flow through the stent 102 when it is in an expanded state. In some embodiments the interior lumen 109 has a generally uniform cross-sectional area along the length of the stent. In one embodiment of the present disclosure, the stent 102 may have a diamond lattice or any suitable pattern. Further, stent 102 may have a uniform diameter along its length, or may be tapered at the distal end to allow convenient insertion within the body. Depending upon the particular implementation and intended use, the length of the stent 102 may vary. The diameter of the stent 102 may be tailored to the diameter of the body lumen. Similarly, depending upon the particular implementation and intended use, the stent 102 can be rigid along its entire length, flexible along a portion of its length, or configured for flexure at only certain specified locations.

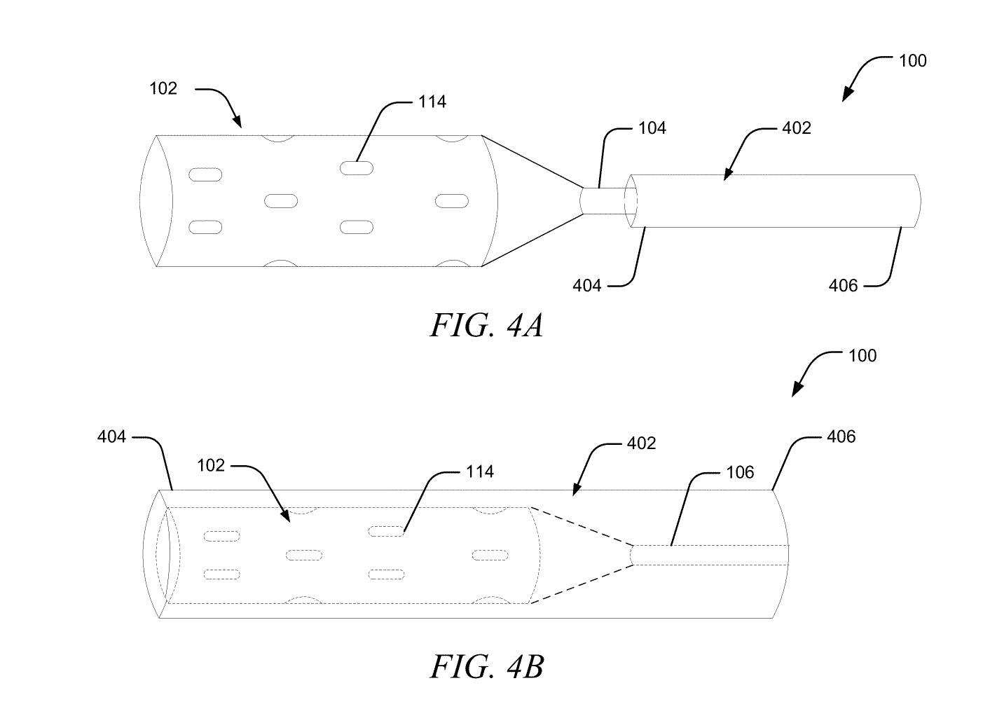

The stent 102 may be implemented as an expandable device made of a smooth material that is sufficiently flexible to conform to the body lumen while at the same time being sufficiently rigid to position the electrodes 114 against the vessel wall with a uniform and gentle pressure. Once appropriately deployed, the stent 102 expands to conform to the blood vessel shape, facilitating appropriate positioning. The stent 102 may be self-expanding or may expand by known mechanisms. These expansion mechanisms are discussed in detail in the following section in connection with FIGS. 4A and 4B.

The stent 102 may be made of any suitable material that is compatible with living tissue or a living system, non-toxic or non-injurious, and does not cause immunological reaction or rejection. Such materials may include, for example, polymers, nitinol, ePTFE, fabric, and suitable nickel and titanium alloys. For example, stent 102 can be made from polyurethane that is non-electrically conductive and biocompatible. In general, stent 102 may be formed of a material that is sufficiently flexible to conform to the bodily location in which it is employed, yet sufficiently rigid to maintain the integrity of lumen 109.

One or more electrodes 114 may be attached to the outer surface of the stent 102. In the illustrated embodiment, the electrodes 114 are an electrode (e.g., radio frequency electrode) configured as a cube or a cuboidal member having regular or irregularly outer surface. In other embodiments, the electrodes have a circular or oblong shape. In one embodiment, the stent 102 may be formed with recesses within which the electrodes 114 may be mounted. It should be understood that each of the electrodes 114 can be configured as a disc, a plate, a strut, a ring, or other suitable configuration, as desired.

The electrodes 114 may be disposed on the stent in any desired manner. For example, the electrodes 114 may be arranged in a staggered configuration or aligned around a circumference of the stent 102. In addition, the number of electrodes may vary depending on the target area, and the condition being treated. For example, there may be two, three or more electrodes. As will be recognized, other number of electrodes 114 may also be contemplated. Further, electrodes 114 can be formed using any conductive, biocompatible material. Examples of suitable material include metals, alloys, conductive polymers, and conductive carbon. In one example arrangement of electrodes 114, the electrodes are arranged to treat the vessel wall such that any longitudinally extending line drawn along the vessel wall in the area treated passes through the ablative zone of at least one electrode, and the plurality of electrodes 114 are also spaced from each other such that there are gaps between the ablative zones of the electrodes 114. Such an arrangement may ensure that the generally longitudinally extending nerves along the renal artery are treated while avoiding undue weakening of the vessel wall.

In addition, a control and power element (not shown), located at the proximal end of the system 100, may be coupled to the electrodes 114 through connectors 116 to provide the necessary electrical energy to activate the electrodes 114. The connectors 116 may be conductive wires, for example. The electrodes 114 may be configured to operate at a frequency of approximately 460 kHz. It is contemplated that any desired frequency may be used, for example, in the RF range, from 450-500 kHz. However, it is also contemplated that different types of energy outside the RF spectrum may be used as desired, for example, but not limited to ultrasound, microwave, laser, and thermal energy.

Arrangement of electrodes 114 on the surface of the stent 102 may be optimized to produce the desired therapeutic ablative effect. Thus, the size, spacing, and placement of electrodes 114 will vary based on the application of RF energy to surrounding tissues. In one embodiment, electrodes 114 may be arranged to preclude overlap among the RF fields produced by the individual electrodes. That arrangement ensures a relatively uniform application of energy to the perivascular nerve tissues.

The electrodes 114 conduct electrical current pulses to stimulate nerve fibers, muscle fibers, or other body tissues. In one embodiment, the control element may control the activation, timing and electrical characteristics of the modulation system 100. For example, the control element can, if desired, control one or more of the timing, frequency, strength, duration, and waveform of the pulses. In addition, the control element can selectively activate one or more electrodes 114 for stimulation.

In at least some embodiments, one or more sensors (depicted in FIG. 1 by reference numeral 120) may be located on the outer surface of the stent 102 or proximate the electrodes 114. These sensors may be connected to the control element or other monitoring device to monitor one or more conditions (e.g., pressure, temperature, or impedance) surrounding the stent 102, the electrodes 114, or the blood and/or luminal surface of the blood vessel proximate the site of ablation.

The embodiments of the present disclosure ensure that only target tissues are ablated and surrounding tissues are protected from thermal energy and provide thermal protection for blood. Different techniques may be employed by different embodiments of the present disclosure to achieve this purpose. In one embodiment of the present disclosure the stent 102 may be made of a non-conductive material that ensures that ablation energy is transferred only to tissue proximate the electrodes 114. Suitable non-conductive materials may be used for manufacturing the stent 102. For example, known polymers may be used such as polyurethane that is non-electrically conductive and biocompatible.

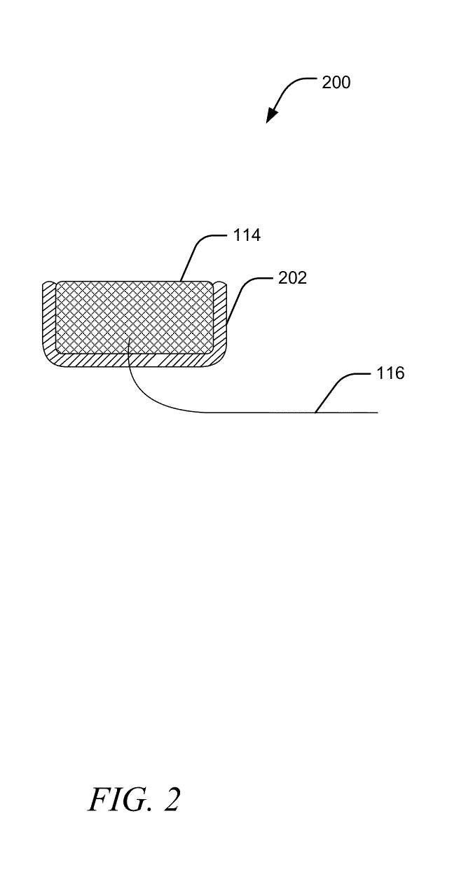

In another embodiment of the present disclosure, each electrode 114 may include an insulation layer between the electrode and the stent 102. FIG. 2 is a side cross-sectional view of an electrode 114 having an insulation layer 202. As shown, the insulation layer 202 surrounds the electrode 114 to electrically isolate the electrode 114 from the stent 102 while leaving an exposed surface to allow the energy provided through the electrode 114 to ablate. An electrode 114 as shown in this figure will generally be mounted on a stent 102 with the exposed surface of the electrode 114 facing outwardly and such an electrode will also preferably be mounted so that the exposed surface can be positioned against the vessel wall in the treatment zone.

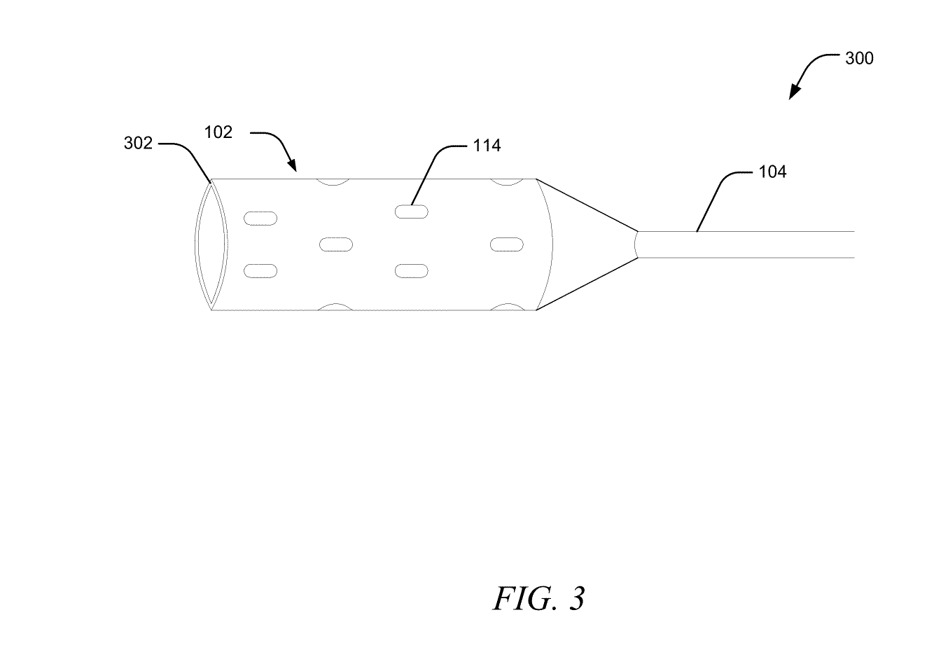

FIG. 3 illustrates an alternative embodiment of the renal nerve modulation system 300. Here, the entire outer surface of the stent 102 may include an insulation layer 302. In each of the embodiments shown in FIGS. 2 and 3, insulation may be provided by mechanisms known to those skilled in the art. Suitable material to manufacture the insulation layer 202, 302 may include Teflon, or other known polymers. The electrodes 114 are mounted on the insulation layer over the stent 102. In this embodiment, the stent may be made from a conductive material such as stainless steel or nitinol and yet be kept electrically isolated from the electrodes 114.

Another aspect of the present disclosure is that in some embodiments the element providing ablation is expandable in nature. FIG. 4A is a distal end of the renal nerve modulation system 100 in an expanded state, while FIG. 4B is a distal end of the system 100 in a collapsed or compressed state. For state change purposes, the embodiments of the present disclosure employ a sheath 402 for enclosing the stent 102.

The sheath 402 may define a substantially circular hollow lumen, having a proximal end 406 and a distal end 404, adapted to deploy the stent 102 within a patient's body. The sheath 402 exerts a radially inwardly directed pressure on the stent 102 keeping it in the compressed state, as shown in FIG. 4B. Once the stent 102 exits the sheath 402; however, the pressure is released, and the stent 102 expands, as shown in

FIG. 4A. It will be understood that in such situations, the material and thickness of the sheath 402 is selected such that it is capable of withstanding a greater force than the force exerted by the stent 102 on the sheath 402. If the sheath 402 material is too thin or too elastic, it may not be sufficient to hold the stent 102 in the compressed state and the stent 102 may expand within the sheath 402. Alternatively, if the sheath 402 is too rigid or thick, it may not be able to traverse the circuitous vasculature path, causing injury to the vessel walls. Therefore a suitable material is preferably chosen with a thickness keeping both aspects in mind.

In another embodiment, one or more pull wires (not shown) are used to expand or collapse the stent. Pull wires may be attached to the stent's outer surface at one or more positions. In one embodiment, when a pull wire is pulled or pushed it exerts a force on the stent 102 in to keep the stent in a compressed state. When the pull string is released, the force is released allowing the stent 102 to expand. Moreover, means to pull, push, or release the pull wire may be provided at the proximal end of the system 100 allowing operators to easily expand or compress the stent 102, as required. In addition, the amount of expansion and compression may also be controlled.

Various mechanisms to change the state of the stent 102 may be contemplated. In one embodiment, the stent 102 may be made from a self-expandable material. For example, such members may be formed of shape memory alloys such as Nitinol or any other self-expandable material commonly known in the art.

Alternative expansion mechanisms may be applied without departing from the scope of the present disclosure. The stent 102 may, for example, be expanded by an inflation mechanism that exerts an outward radial force on the stent 102 to expand it. Such inflating mechanism (not shown) may include one or more balloons inflated by fluids, or dilators. Other such inflating means may include springs, or levers.

The expansion of the stent 102 should be such that is does not damage the artery by exerting too large a force on the vessel walls. For example, each of the electrodes 114 may exert approximately 5-10 grams of force on the vessel wall, avoiding vessel damage. In some embodiments, the stent 102 may include visualization devices such as a camera. The stent 102 may be provided with a fluorescent dye to make it easier to visualize the extent of expansion. Further, the stent 102 may include a force or expansion-limiting component that prevents the stent 102 from expanding beyond a certain limit. For example, the diameter of the stent 102 may be maintained below 6-7 mm. Often, the expansion limit may be set during manufacture of the stent 102. In general, operators may know the average size of renal arteries, and they may ensure that the stent 102 does not expand beyond the average artery size.

The expansion of the stent 102 may also assist in pushing the electrodes 114 against the vessel walls to provide effective ablation. For example, the diameter of the stent 102 may be sized such that when the stent 102 is fully expanded within the lumen of the vessel, the stent 102 exerts a force on the vessel wall to ensure generally uniform circumferential contact of the vessel wall, and thus urge the electrodes 114 against the luminal surface of the vessel wall. In other embodiments, the stent may be sized such that expansion of the stent 102 positions the electrodes 114 at a predetermined distance from the vessel wall to maintain the electrodes in spaced relationship with the vessel wall.

It should be noted that the stent 102 is designed for retraction from the patient's body after the treatment is concluded. To that end, the stent 102 reverses the procedure set out above, by first collapsing the stent body and then retracting it into sheath 402, employing control wires or other suitable conventional means.

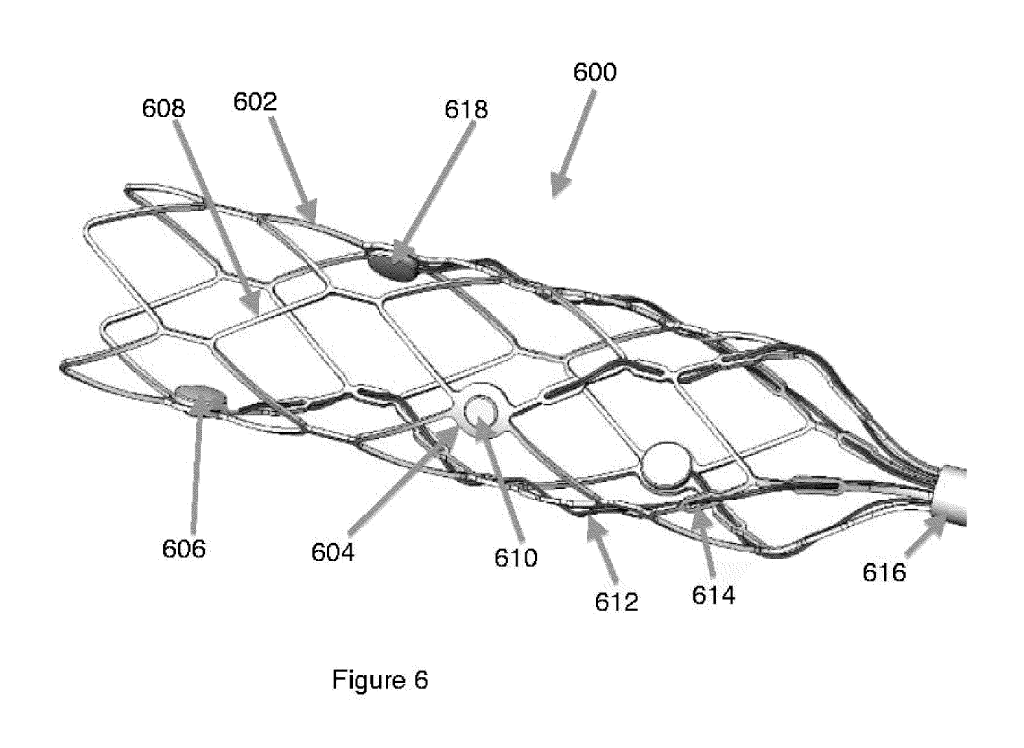

FIG. 6 illustrates the distal portion of an example embodiment of a renal nerve modulation system 600. A stent 602 may include mounts 604 for affixed electrodes 606 thereon. The stent 602 may have a lattice-work pattern of interconnected struts 608 as shown or another suitable self-expanding pattern as is known in the art. The stent 602 may be biased to the expanded position shown and may be made from a resilient material such as described above. The stent 602, particularly if electrically conductive, may be electrically isolated from the electrodes 606. The mounts may be located at selected interstices of the struts 608 or may be located along an individual strut. The mounts may be annular as shown or may have another suitable shape to provide geometry for an electrode to be securely fixed thereto. In some contemplated embodiments, the stent 602 lacks mounts and the electrodes 606 are shaped to be fixed securely to the stent 602. For example, an electrode may be provided with a Y-shaped groove to allow it to be securely fixed to an interstice of the illustrated stent 602. Electrodes 606 are shown as having a circular pad 610 which contacts the vessel wall and a mounting portion 618. Electrode 606 can be described as a smaller disc (pad 610) centered on a larger disc (mounting portion 618). It can be appreciated that pads of various sizes and shapes are contemplated. For example, electrodes may be provided that have oblong or oval shaped pads. Four electrodes 606 are shown. It can be appreciated that more or fewer electrodes may be included. For example, 3, 4, 5, 6, 7, 8, 9, 10 or more pads may be includes in various embodiments. The electrodes are preferably spaced from each other and distributed to provide good circumferential coverage. Each electrode may be connected to a power source by a conductor 612 such as a wire. The stent 602 may be provided with features such as slots 614 to allow the conductors 612 to be securely attached. The proximal end of the stent 602 is preferably securely attached to an elongate member 616 (the distal end of which is illustrated). In this embodiment, elongate member 616 is a tube having a lumen and the proximal ends of the stent 602 are fixed within the lumen and the conductors 612 extend through the lumen. The lumen may also include sufficient room for a guidewire.

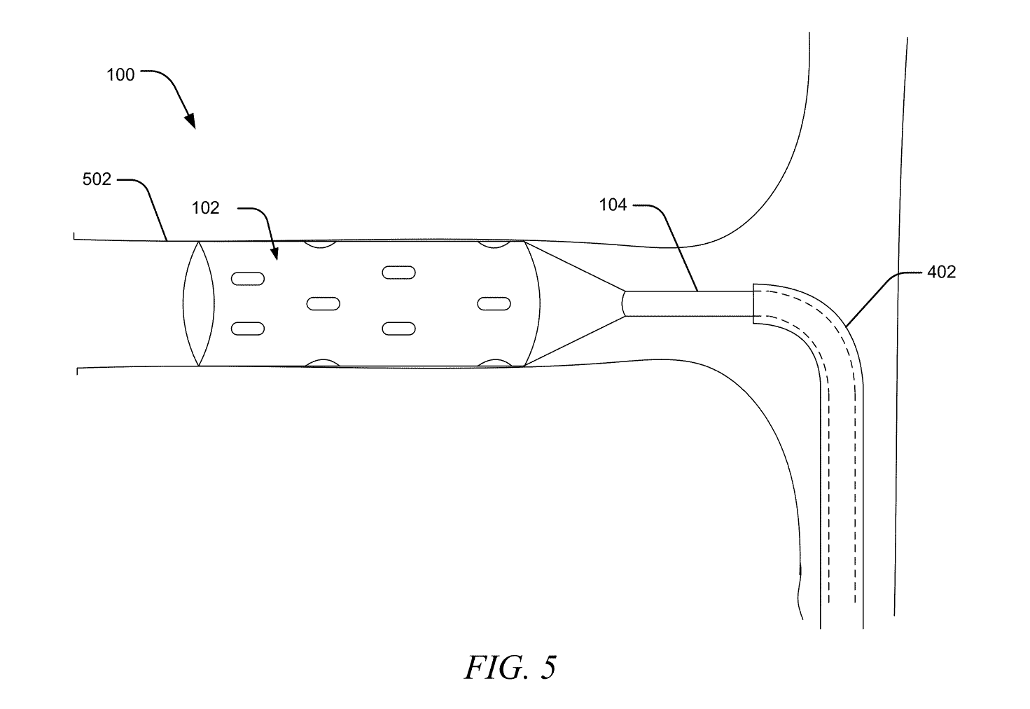

FIG. 5 illustrates a method of ablating tissue using the renal nerve modulation system 100, shown in FIG. 1. The system 100 may be introduced percutaneously as is conventional in the intravascular medicinal device arts. For example, a guidewire may be introduced percutaneously through a femoral artery and navigated to a renal artery using standard radiographic techniques. In the present method the sheath 402 is first introduced over the guide wire, after which the guide wire is withdrawn. Subsequently, the stent 102 is introduced into the sheath 402. Alternatively, the sheath 402 carrying the stent 102 in the compressed state may be introduced over the guide wires.

Once the sheath 402 reaches the desired location proximate a vessel wall 502 where ablation is required, the sheath 402 may be retracted proximally to allow the stent 102 to expand or the stent 102 may be urged distally to extend beyond the distal end of the sheath 402. In the shown embodiment the stent 102 expands to circumferentially contact the luminal surface of the vessel wall 502.

The electrodes 114 may then be activated to ablate the desired nerve tissue. To allow ablation of only the target tissue while protecting surrounding tissue from thermal energy, the stent 102 may include varying configurations and may include means for selectively activating some of the electrodes. The electrodes 114 may be activated sequentially, simultaneously, or selectively, as desired. During this procedure, the system 100 may continuously monitor the temperature or impedance at the electrodes 114 and the vessel wall 502. Known radiography techniques may be utilized to monitor the tissue being ablated. Once the tissue is sufficiently ablated, the sheath 402 may be advanced or the stent 102 retracted to compress the stent 102 within the sheath 402, and subsequently the sheath 402 may be retrieved from the patient's body.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A medical device for nerve modulation through the wall of a blood vessel, comprising:
an elongate member having a proximal end and a distal end, and
a hollow cylindrical ablation member connected to the distal end of the elongate member, wherein the ablation member is provided with a plurality of electrodes distributed over its outer surface;
wherein the ablation member is configured to be shift between a collapsed state in which it is moveable through a lumen of a blood vessel and an expanded state in which at least a portion of the ablation member is placed in contact with or spaced from the wall of the blood vessel.

2. The medical device of claim 1, wherein the ablation member is self-expanding.

3. The medical device of claim 1, wherein the ablation member is expanded by an actuating means.

4. The medical device of claim 1, wherein the ablation member comprises a stent to which electrodes have been secured.

5. The medical device of claim 1, wherein the ablation member includes one or more insulated sections.

6. The medical device of claim 5, wherein the one or more insulated sections includes an insulating layer covering the outer surface of the ablation member.

7. The medical device of claim 5, wherein the electrodes are configured to preclude overlap among the fields produced by the electrodes.

8. The medical device of claim 1, wherein the ablation member is made of a non-conductive material.

9. The medical device of claim 1 further comprising one or more sensors on the ablation member.

10. The medical device of claim 1, wherein the electrodes are coupled to one or more actuators for activating one or more of the electrodes.

11. A system for nerve modulation through the wall of a blood vessel, the system comprising:
a catheter sheath having a proximal end, a distal end, and a lumen extending from the proximal to the distal end;
an elongate member extending within the lumen of the catheter sheath, the elongate member having a proximal end and a distal end, and
an ablation member coupled to the distal end of the elongate member, the ablation member having a plurality of electrodes disposed on the outer surface of the ablation member, and the ablation member being substantially cylindrical and being configured to shift between a non-expanded configuration and an expanded configuration.

12. The system of claim 11, wherein the ablation member comprises a lattice-work tubular member and wherein the ablation member is configured to shift to the expanded configuration by extending the ablation member out of the distal end of the catheter sheath.

13. The system of claim 11, wherein the ablation member is self-expanding.

14. The system of claim 11, wherein the ablation member comprises a stent to which electrodes are secured.

15. The system of claim 11, wherein the ablation member includes one or more insulated sections.

16. The system of claim 15, wherein the one or more insulated sections comprises an insulated layer covering the outer surface of the ablation member.

17. The system of claim 15, wherein the one or more insulated sections at least partially surround the electrodes.

18. The system of claim 15, wherein each electrode has a bottom surface, a top surface and a side surface extending around a perimeter of the electrode between the bottom surface and the top surface and wherein the one or more insulated sections surround each electrode bottom and side surfaces.

19. The system of claim 11, further comprising one or more sensors mounted on the ablation member.

20. A method for ablating a nerve through a blood vessel, the method comprising:
advancing a medical device to a desired location in a blood vessel lumen, the medical device including:
an elongate member having a proximal end and a distal end;
a hollow cylindrical ablation member disposed at the distal end of the elongate member, wherein the ablation member includes a plurality of electrodes positioned on its outer surface, and the ablation member being configured to shift between a collapsed state and an expanded state;
expanding the ablation member in the vessel lumen; and
activating one or more electrodes to ablate at least a portion of the nerve.
